(19) United States
(12) Patent Application Publication
(10) Pub. No.: US 2021/0283046 A1
(43) Pub. Date: Sep. 16, 2021

(54) PHARMACEUTICAL DOSAGE FORM WHICH CAN BE ADMINISTERED ORALLY AND HAS MODIFIED RELEASE

(71) Applicants: Bayer Aktiengesellschaft, Leverkusen (DE); Bayer Pharma Aktiengesellschaft, Berlin (DE)

(72) Inventors: Anke Stroyer, Berlin (DE); Carmen Lobback, Schöneiche (DE); Peter Serno, Bergisch Gladbach (DE); Tia Jacobs, Wuppertal (DE)

(73) Assignees: Bayer Aktiengesellschaft, Leverkusen (DE); Bayer Pharma Aktiengesellschaft, Berlin (DE)

(21) Appl. No.: 17/262,491

(22) PCT Filed: Jul. 19, 2019
(86) PCT No.: PCT/EP2019/069564

§ 371 Date: Jan. 22, 2021

(30) Foreign Application Priority Data
Jul. 24, 2018 (EP) 18185135.3

(51) Int. Cl. A61K 9/00; A61K 31/196; A61K 9/20; A61K 9/28

(52) U.S. Cl. CPC A61K 9/0004; A61K 31/196; A61K 9/2027; A61K 9/28; A61K 9/205; A61K 9/2095

(57) ABSTRACT

The present invention relates to orally administrable modified-release pharmaceutical dosage forms comprising (3S)-3-(4-chloro-3-{[(2S,3R)-2-(4-chlorophenyl)-4,4,4-trifluoro-3-methylbutanoyl]amino}phenyl)-3-cyclopropylpropanoic acid and to processes for producing the dosage forms and to the use thereof for the treatment and/or prevention of diseases, in particular for the treatment and/or prevention of cardiac, renal, pulmonary and ophthalmological disorders, disorders of the central nervous system, fibrotic and inflammatory disorders and metabolic disorders.

The present invention relates to orally administrable modified-release pharmaceutical dosage forms comprising (3S)-3-(4-chloro-3-{[(2S,3R)-2-(4-chlorophenyl)-4,4,4-trifluoro-3-methylbutanoyl]amino}phenyl)-3-cyclopropylpropanoic acid and to processes for producing the dosage forms and to the use thereof for the treatment and/or prevention of diseases, in particular for the treatment and/or prevention of cardiac, renal, pulmonary and ophthalmological disorders, disorders of the central nervous system, fibrotic and inflammatory disorders and metabolic disorders.

WO 2012/139888 discloses the compound (3S)-3-(4-chloro-3-{[(2S,3R)-2-(4-chlorophenyl)-4,4,4-trifluoro-3-methylbutanoyl]amino}phenyl)-3-cyclopropylpropanoic acid of the formula (I)

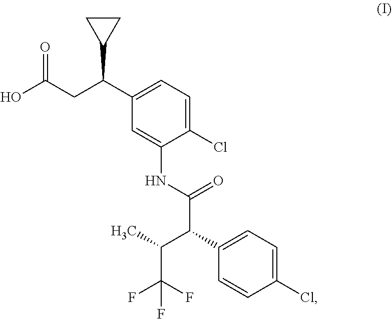 [SMILES: C[C@H]([C@H](C(=O)Nc1cc([C@@H](CC(=O)O)C2CC2)ccc1Cl)c1ccc(Cl)cc1)C(F)(F)F]

and the preparation thereof, in Example 22. The compound of the formula (I) acts as activator of soluble guanylate cyclase. The document also discloses that the chemical compounds described can generally be converted into tablets, orally administrable suspensions and orally administrable solutions. These pharmaceutical dosage forms are exclusively rapid-release pharmaceutical compositions.

For diseases that require treatment over a longer period or in the long-term prophylaxis of diseases, it is desirable to keep the dosing frequency of medicaments as low as possible. This is not only more convenient for the patient, it also increases the reliability of treatment by reducing the disadvantages of irregular dosing. The desired reduction in dosing frequency, for example from administration twice a day to once a day, can be achieved by prolonging the therapeutically effective plasma levels through modified release of active ingredients from dosage forms.

Moreover, after dosing with dosage forms with modified active ingredient release, it is possible for side effects to be reduced through smoothing of the plasma level time curve. By minimizing the peak-trough ratio, i.e. by avoiding high plasma active ingredient concentrations, which are frequently observed after administration of rapid-release pharmaceutical forms, the occurrence of unwanted side effects correlating with the concentration peaks can be reduced. A modified-release pharmaceutical form should therefore be developed. An osmotic release system was chosen for this in order to ensure the required profile of even, long-lasting and complete release of active ingredient over a variable, predefined time period. Compared to other delayed-release drug delivery systems, osmotic release systems are characterized, for example, in that the release profiles can be flexibly adjusted by adjusting the thickness of the shell (Kaushal, A. M., Garg, S. An Update on Osmotic Drug Delivery Patents. Pharmaceutical Technology. 2003. 13(1):8-97).

Osmotic release systems are also referred to as gastrointestinal therapeutic systems (GITS) or oral osmotic systems (OROS). The long-lasting and even release of an active ingredient is controlled by the osmotic pressure.

Osmotic release systems can be differentiated into single-chamber systems (elementary osmotic pump) and two-chamber systems (push-pull systems).

In single-chamber systems, one or more osmotically active substances are mixed with the active ingredient and pressed into cores. These cores are surrounded by a semipermeable membrane that has at least one orifice. This semipermeable membrane, hereinafter referred to as the shell, is impermeable to components of the core, but allows ingress of water from outside by osmosis. The water that has penetrated in then releases the active ingredient in dissolved or suspended form from one or more orifices in the shell via the osmotic pressure that develops. The overall release of active ingredient and rate of release can be largely controlled via the thickness and porosity of the shell, the composition of the core and the number and size of the orifices.

In two-chamber systems, one chamber comprises the active ingredient and the other chamber comprises the osmotically active substance. The two chambers may be separated by a flexible separating wall. This core is likewise surrounded by a shell that has at least one orifice on the side of the chamber containing the active ingredient.

Advantages, formulation aspects, use forms and information on production processes for osmotic release systems are described inter alia in the following publications:
Kaushal, A. M., Garg, S.: “An Update on Osmotic Drug Delivery Patents”, Pharmaceutical Technology 2003, 13, 8-97.
Kumar, P. and Mishra, B.: “An Overview of Recent Patents on Oral Osmotic Drug Delivery Systems”, Recent Patents on Drug Delivery & Formulation 2007, 1, 236-255.
Verma, R. K., Mishra, B., Garg, S.: “Osmotically controlled oral drug delivery”, Drug Development and Industrial Pharmacy 2000, 26, 695-708.
Verma, R. K., Krishna, D. M., Garg, S.: “Formulation aspects in the development of osmotically controlled oral drug delivery systems”, Journal of Controlled Release 2002, 79, 7-27.
Sareen. R., Jain, N., Kumar, D.: “An Insight to Osmotic Drug Delivery”, Current Drug Delivery 2012, 9, 285-296.
Malaterre, V., Ogorka, J., Loggia, N., Gurny, R.: “Oral osmotically driven systems: 30 years of development and clinical use”, European Journal of Pharmaceutics and Biopharmaceutics 2009, 73, 311-323.
U.S. Pat. No. 4,327,725
U.S. Pat. No. 4,765,989
US 20030161882
EP-A 1024793

In the context of the present invention, the compound of the formula (I) should be formulated in the form of an osmotic release system in order to achieve long-lasting and even release.

The hydrophilic swellable polymer usually employed is polyethylene oxide, particularly in the case of two-chamber systems (WO 2006/072367). Unexpectedly, it is not possible for the compound of the formula (I) to be formulated in the customary manner in the form of an osmotic release system with polyethylene oxide as hydrophilic swellable polymer. During the production process of the osmotic release system comprising the compound of the formula (I), melt phenomena were encountered during granulation. The resulting inefficient production process yielded dosage forms that did not meet the requirements and the specification of a pharmaceutical product.

When using the compound of the formula (I) and polyethylene oxide as hydrophilic swellable polymer, a change in the consistency of part of the granulate obtained was observed during dry granulation with a roller. The granulate components fused to one another to form a hard plastic-like mass similar to a solidified melt, which was not suitable for further processing. The planned production process had to be abandoned. Comminution of the solidified melt by grating and sieving was possible only with high expenditure of force, material and time, which rendered the production process inefficient and unreliable with regard to reproducible pharmaceutical product quality.

During further processing of the laboriously-sieved, active ingredient-comprising roller granulate, further disadvantageous effects occurred when pressing the tablets. Even while still in the infeed hopper, “bridging” was observed, which is when the individual granules become caught on one another as a consequence of the rough surface of the granules. As a result of this, the tableting mixture was not free-flowing without additional agitation. This meant that the granulate could not be used as a tableting mixture for continuous tableting. Here too, the production process had to be abandoned. There was substantial adherence of the tableting mixture to tableting machine components such as punches, dies and rotary table. The few tablets obtained showed a tendency to “capping”, which is when the upper or lower part of the tablet, on ejection from the tablet press or during processing, have become fully or partially detached from the main part and formed a cap. Such tablets do not meet the requirements of acceptable pharmaceutical quality and are no longer suitable for use.

Various samples were collected of the active ingredient-comprising powder mixture prior to granulation, of the plastic-like mass prior to sieving, of the plastic-like mass after comminution and sieving and of the residue left on the sieve used for milling and their analysis showed substantial variation in the content of the compound of the formula (I). Starting from 100% of the declared active ingredient content in the active ingredient-comprising powder mixture prior to granulation, the samples showed content values from 107% to 120% based on the declared active ingredient content. The consistently elevated content values are probably due to the fact that, during production, only some of the roller granules melt and the compound of the formula (I) is present in heterogeneously dispersed form. A pharmaceutical dosage form having such deviations in the active ingredient content is unacceptable and cannot be used for further development. It can be assumed that the measured variation in the content of the powder mixture also lead to variation in the content of a tablet produced therefrom and that such tablets accordingly will not meet pharmacopoeial requirements, for example those for content uniformity (Ph. Eur. 9th edition; 2.9.40 “Uniformity of Dosage Units”).

By replacing the hydrophilic swellable polymer polyethylene oxide with a hydrophilic swellable polymer suitable according to the invention, it is surprisingly possible to obtain an osmotic release system that has neither the described disadvantageous properties of the osmotic release system comprising the compound of the formula (I) and polyethylene oxide nor the described disadvantages in the production of the osmotic release system comprising the compound of the formula (I) and polyethylene oxide. Hydrophilic swellable polymers suitable according to the invention are preferably selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer, polyacrylic acids and salts and mixtures of the individual polymers. When the compound of the formula (I) was used together with hydrophilic polymers suitable according to the invention, no melt phenomena occur and no other disadvantageous observations are made during the individual steps in production. The production process can be completed without any unplanned interruptions. The content determination gives results that meet the specification for the declared active ingredient content.

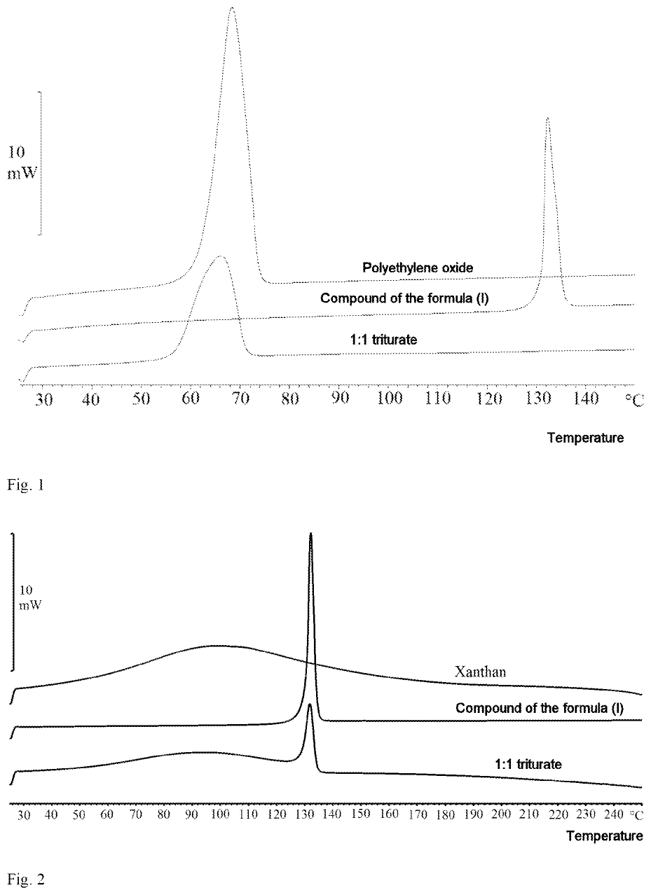

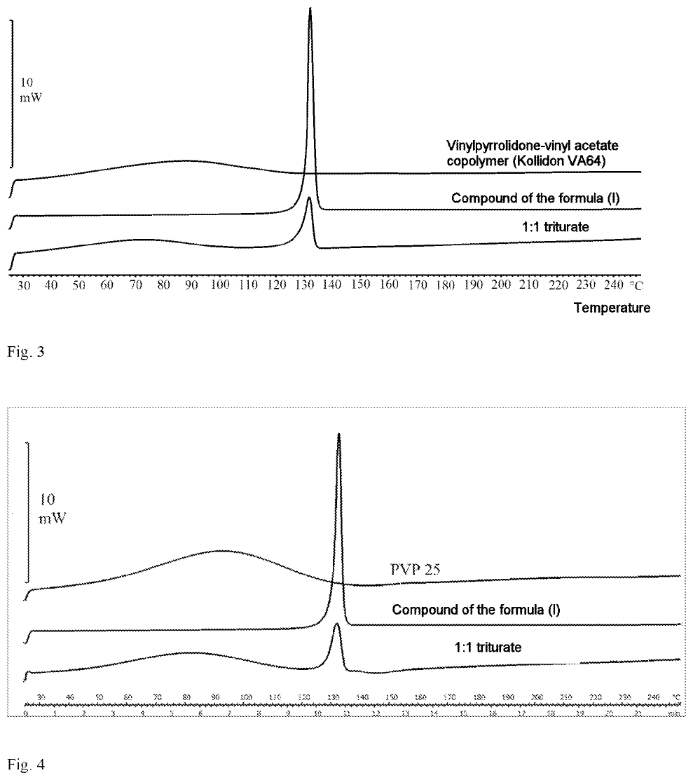

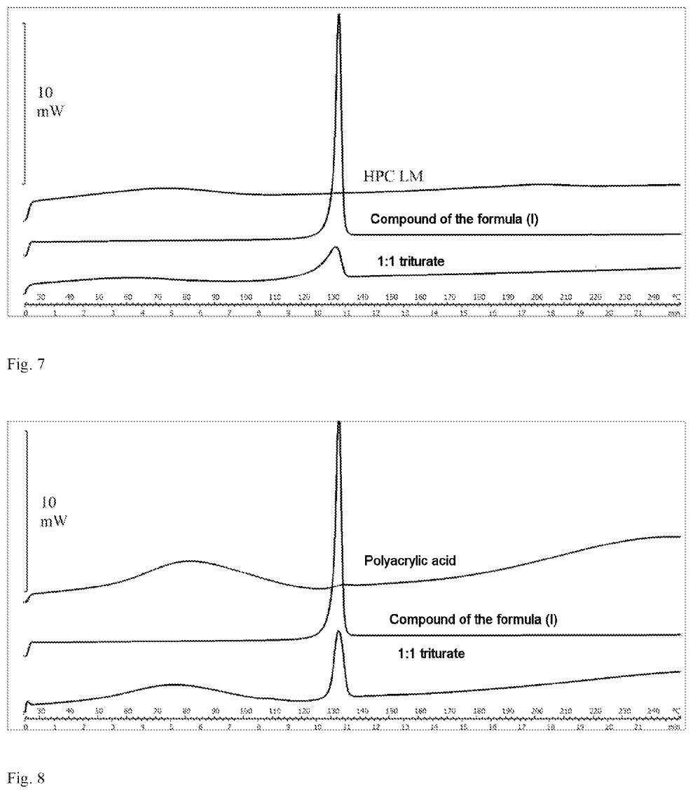

The different behaviour of a mixture of the compound of the formula (I) and polyethylene oxide compared to a mixture of the compound of the formula (I) and hydrophilic polymers suitable according to the invention can additionally be demonstrated by measuring the DSC (differential scanning calorimetry) thermograms of the respective substances alone and in 1:1 triturates (binary mixtures). The changes observed in the thermograms are indicative of the processability of the powder mixture. A triturate comprising equal parts of the compound of the formula (I) and polyethylene oxide shows no melting peak that can be assigned to the compound of the formula (I) (FIG. 1). Disappearance of the melting peak of the active ingredient, broadening of the melting peak of the hydrophilically swellable polymer and an earlier onset of melting correlate with the described processing deficiencies. The melting process accordingly already begins at a temperature between 50° C. and 60° C. Such temperatures can develop during production of the osmotic release systems, giving rise to the described melt phenomena. A triturate comprising equal parts of the compound of the formula (I) and xanthan shows, in addition to the melting peak of xanthan, an additional melting peak that can be assigned to the compound of the formula (I) (FIG. 2). Binary mixtures of the compound of the formula (I) with vinylpyrrolidone-vinyl acetate-copolymer (Kollidon VA64), polyvinylpyrrolidone (PVP 25), hydroxypropylcellulose (HPC LM), anionic copolymers of methacrylic acid and methyl methacrylates (Eudragit L100, Eudragit RL PO) likewise show a melting peak which is attributable to the compound of the formula (I) and is in a high temperature region (FIG. 3 to FIG. 7). Since these polymers are present in amorphous form, no melting peak attributable to the polymers is detected. The thermogram of a triturate of the compound of the formula (I) and polyacrylic acid shows no melting peak that can be assigned to the compound of the formula (I) (FIG. 8). It can be assumed that the compound of the formula (I) dissolves with increasing temperature after the glass transition temperature of polyacrylic acid. Since the glass transition temperature is approx. 106° C., melt phenomena during production of an osmotic release system with polyacrylic acid as hydrophilic swellable polymer are not to be expected.

The present invention provides a solid, orally administrable modified-release pharmaceutical dosage form comprising (3S)-3-(4-chloro-3-{[(2S,3R)-2-(4-chlorophenyl)-4,4,4-trifluoro-3-methylbutanoyl]amino}phenyl)-3-cyclopropylpropanoic acid of the formula (I), characterized in that 80% of the compound of the formula (I) is released over a period of 2 to 24, preferably 4 to 20, hours, measured according to the USP dissolution method (USP 39; chapter <711> Dissolution) using Apparatus 2 (paddle) and the instructions under “Dissolution profile”

Two-chamber systems (push-pull systems) and single-chamber systems (elementary osmotic pump) are both suitable for formulating the compounds of the formula (I) in the form of an osmotic release system. The two-chamber system and the single-chamber system both consist of a core coated with a shell and optionally with an outer coating. In the osmotic release systems, the compound of the formula (I) may be present either in crystalline or amorphous form or in mixtures having crystalline and amorphous fractions.

The compound of the formula (I) is preferably present in crystalline form in the osmotic release system. The compound of the formula (I) is preferably present in micronized form in the osmotic release system.

The present invention provides a solid, orally administrable modified-release pharmaceutical dosage form comprising (3S)-3-(4-chloro-3-{[(2S,3R)-2-(4-chlorophenyl)-4,4,4-trifluoro-3-methylbutanoyl]amino}phenyl)-3-cyclopropylpropanoic acid of the formula (I), characterized in that the pharmaceutical dosage form is based on an osmotic release system.

The present invention provides a solid, orally administrable modified-release pharmaceutical dosage form comprising the compound of the formula (I), characterized in that the pharmaceutical dosage form is based on an osmotic single-chamber system.

The present invention provides a solid, orally administrable modified-release pharmaceutical dosage form comprising the compound of the formula (I), characterized in that the pharmaceutical dosage form is based on an osmotic two-chamber system.

In one embodiment, the osmotic release system consists of a core and a shell, wherein the shell consists of a water-permeable material impermeable to the components of the core and has at least one orifice and wherein the core comprises the compound of the formula (I) and at least one hydrophilic swellable polymer.

In a further embodiment, the osmotic release system consists of a core and a shell, wherein the shell consists of a water-permeable material impermeable to the components of the core and has at least one orifice and wherein the core comprises the compound of the formula (I) and at least one hydrophilic swellable polymer preferably selected from a list consisting of xanthan, cellulose derivatives, for example hydroxypropylcellulose, hydroxypropyl methylcellulose, sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or preferably selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer and polyacrylic acids, more preferably xanthan and vinylpyrrolidone-vinyl acetate copolymer or a mixture of xanthan and vinylpyrrolidone-vinyl acetate copolymer.

In a further embodiment, the osmotic release system consists of a core and a shell, wherein the shell consists of a water-permeable material impermeable to the components of the core and has at least one orifice and wherein the core comprises the compound of the formula (I), at least one hydrophilic swellable polymer preferably selected from a list consisting of xanthan, cellulose derivatives, for example hydroxypropylcellulose, hydroxypropyl methylcellulose, sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or preferably selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer and polyacrylic acids, more preferably xanthan and vinylpyrrolidone-vinyl acetate copolymer or a mixture of xanthan and vinylpyrrolidone-vinyl acetate copolymer, optionally at least one pharmaceutically customary excipient and optionally an osmotically active additive.

In a further embodiment, the osmotic release system consists of a core and a shell, wherein the shell consists of a water-permeable material impermeable to the components of the core and has at least one orifice and wherein the core comprises the compound of the formula (I), the hydrophilic swellable polymer xanthan, optionally at least one further hydrophilic swellable polymer, optionally at least one pharmaceutically customary excipient and optionally an osmotically active additive.

In a further embodiment, the osmotic release system consists of a core and a shell, wherein the shell consists of a water-permeable material impermeable to the components of the core and has at least one orifice and wherein the core comprises 0.5% by weight to 50% by weight of the compound of the formula (I), 40% by weight to 99.5% by weight of at least one hydrophilic swellable polymer preferably selected from a list consisting of xanthan, cellulose derivatives, for example hydroxypropylcellulose, hydroxypropyl methylcellulose, sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or preferably selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer and polyacrylic acids, more preferably xanthan and vinylpyrrolidone-vinyl acetate copolymer or a mixture of xanthan and vinylpyrrolidone-vinyl acetate copolymer, optionally at least one pharmaceutically customary excipient and optionally an osmotically active additive.

In a further embodiment, the osmotic release system consists of a core and a shell, wherein the shell consists of a water-permeable material impermeable to the components of the core and has at least one orifice and wherein the core comprises 1% by weight to 40% by weight of the compound of the formula (I), 50% by weight to 99% by weight of at least one hydrophilic swellable polymer preferably selected from a list consisting of xanthan, cellulose derivatives, for example hydroxypropylcellulose, hydroxypropyl methylcellulose, sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or preferably selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer and polyacrylic acids, more preferably xanthan and vinylpyrrolidone-vinyl acetate copolymer or a mixture of xanthan and vinylpyrrolidone-vinyl acetate copolymer, optionally at least one pharmaceutically customary excipient and optionally an osmotically active additive.

In a further embodiment, the osmotic release system consists of a core and a shell, wherein the shell consists of a water-permeable material impermeable to the components of the core and has at least one orifice and wherein the core comprises 2% by weight to 20% by weight of the compound of the formula (I), 60% by weight to 90% by weight of at least one hydrophilic swellable polymer, preferably selected from a list consisting of xanthan, cellulose derivatives, for example hydroxypropylcellulose, hydroxypropyl methylcellulose, sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or preferably selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer and polyacrylic acids, more preferably xanthan and vinylpyrrolidone-vinyl acetate copolymer or a mixture of xanthan and vinylpyrrolidone-vinyl acetate copolymer, optionally at least one pharmaceutically customary excipient and optionally an osmotically active additive.

In a further embodiment, the osmotic release system consists of a core and a shell, wherein the shell consists of a water-permeable material impermeable to the components of the core and has at least one orifice and wherein the core comprises 2% by weight to 10% by weight of the compound of the formula (I), 70% by weight to 85% by weight of at least one hydrophilic swellable polymer selected from a list consisting of xanthan, cellulose derivatives, for example hydroxypropylcellulose, hydroxypropyl methylcellulose, sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or preferably selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer and polyacrylic acids, more preferably xanthan and vinylpyrrolidone-vinyl acetate copolymer or a mixture of xanthan and vinylpyrrolidone-vinyl acetate copolymer, optionally at least one pharmaceutically customary excipient and optionally an osmotically active additive.

In a further embodiment, the osmotic release system consists of a core and a shell, wherein the shell consists of a water-permeable material impermeable to the components of the core and has at least one orifice and wherein the core comprises
0.5% by weight to 50% by weight of the compound of the formula (I),
10% by weight to 50% by weight of xanthan,
5% by weight to 40% by weight of a vinylpyrrolidone-vinyl acetate copolymer,
   optionally at least one further hydrophilic swellable polymer, optionally at least one further pharmaceutically customary excipient and optionally an osmotically active additive.

The percentages by weight are in each case based on the total mass of the core.

Preferably, the osmotic single-chamber system comprises as one of the essential constituents of the core the hydrophilic water-swellable polymer xanthan. This is an anionic heteropolysaccharide that is commercially available, for example under the name Rhodigel® (produced by Rhodia) or “Xanthan FN food grade, normal” (produced by Jungbunzlauer Ladenburg GmbH). It is present in an amount of from 10% to 50% by weight, preferably from 25% to 40% by weight, based on the total mass of the core constituents.

A further essential constituent of the core is vinylpyrrolidone-vinyl acetate copolymer. This copolymer is known per se and can be produced with any desired monomer mixing ratios. For example, the commercially available Kollidon® VA64 (produced by BASF) that is preferably used is a 60:40 copolymer. It generally has a weight-average molecular weight, determined by light-scattering measurements, of about 45 000 to about 70 000. The amount of vinylpyrrolidone-vinyl acetate copolymer in the core is 5% to 40% by weight, preferably 15% to 25% by weight, based on the total mass of the core constituents.

Hydrophilic swellable polymers optionally additionally present in the core are, for example, cellulose derivatives, for example hydroxypropylcellulose, hydroxypropyl methylcellulose, sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or salts thereof.

The present invention further provides a process for producing an osmotic release system, characterized in that the components of the core are mixed with one another, granulated and tableted, the resulting core is coated with a shell and the shell is then provided with one or more orifices suitable for the escape of the compound of the formula (I).

The present invention further provides a process for producing an osmotic single-chamber system of the invention, wherein the components of the core are mixed with one another, optionally undergo wet or dry granulation and are then tableted, and the resulting core is coated with the shell. The shell is, on the active ingredient side, provided with one or more orifices. Alternatively, the introduction of the one or more orifices in this process step may be omitted and an outer coating, for example a light-protective coating and/or coloured outer coating, applied first. In this case, it is only after coating with one or more further outer coatings has been carried out that both sides of the tablet are each provided with an orifice extending in each case from the outside as far as the inner core, i.e. traversing the outer coating and the shell and suitable for the escape of the compound of the formula (I).

In a preferred embodiment of the present invention, the core components undergo wet granulation in the production of the osmotic single-chamber system, since this process step achieves better wettability of the constituents of the tablet core, resulting in better penetration of the gastrointestinal fluid into the core, in many cases leading to more rapid and more complete release of the active ingredient.

The shell and optionally present outer coating of the osmotic drug release system of the present invention have at least one orifice or passage through which the active ingredient together with the other core constituents slowly escapes. The orifice is introduced into the shell by laser drilling, mechanical drilling or e.g. by punching. There may be one or more orifices present in the shell. The size of the orifice (diameter) is preferably 0.2 to 1.6 mm, more preferably 0.3 to 1.2 mm. The nature of the orifice and methods for the production thereof are known per se and are described for example in U.S. Pat. Nos. 4,063,064, 4,088,864, 3,916,899 or EP-A 0277092.

In a further embodiment, the core of the osmotic release system consists of two layers, an active ingredient layer and an osmosis layer. An osmotic two-chamber system of this type is described in detail for example in DE 3417113 C2, WO 2006/072367 or WO 2010/060564, the disclosures of which are incorporated herein by reference.

In a further embodiment, the osmotic release system consists of a core and a shell, wherein the shell consists of a water-permeable material impermeable to the components of the core and has at least one orifice and wherein the core consists of an active ingredient layer and an osmosis layer.

In a further embodiment, the osmotic release system consists of a core and a shell, wherein the shell consists of a water-permeable material impermeable to the components of the core and has at least one orifice and wherein the core consists of an active ingredient layer and an osmosis layer and the active ingredient layer comprises 1% by weight to 50% by weight of the compound of the formula (I), 20% by weight to 99% by weight of at least one hydrophilic swellable polymer preferably selected from a list consisting of xanthan, cellulose derivatives, for example hydroxypropylcellulose, hydroxypropyl methylcellulose, sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer and polyacrylic acids, preferably xanthan and vinylpyrrolidone-vinyl acetate copolymer, optionally at least one osmotically active additive and optionally at least one pharmaceutically customary excipient.

In a further embodiment, the active ingredient layer comprises 1% by weight to 45% by weight, preferably 1% by weight to 30% by weight, more preferably 2% by weight to 20% by weight of the compound of the formula (I), 30% by weight to 99% by weight, preferably 50% by weight to 99% by weight, more preferably 60% by weight to 98% by weight, of at least one hydrophilic swellable polymer, optionally at least one osmotically active additive and optionally at least one pharmaceutically customary excipient.

In a further embodiment, the osmotic release system consists of a core and a shell, wherein the shell consists of a water-permeable material impermeable to the components of the core and has at least one orifice and wherein the core consists of one of the active ingredient layers described above and an osmosis layer, wherein the osmosis layer comprises 40% by weight to 90% by weight, preferably 50% by weight to 80% by weight of at least one hydrophilic swellable polymer preferably selected from a list consisting of xanthan, cellulose derivatives, for example hydroxypropylcellulose, hydroxypropyl methylcellulose, sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer and polyacrylic acids, preferably xanthan and vinylpyrrolidone-vinyl acetate copolymer and optionally at least one pharmaceutically customary excipient.

In a further embodiment, the osmotic release system consists of a core and a shell, wherein the shell consists of a water-permeable material impermeable to the components of the core and has at least one orifice and wherein the core consists of an active ingredient layer and an osmosis layer and wherein the active ingredient layer comprises 1% by weight to 50% by weight of the compound of the formula (I), 20% by weight to 99% by weight of at least one hydrophilic swellable polymer preferably selected from a list consisting of xanthan, cellulose derivatives, for example hydroxypropylcellulose, hydroxypropyl methylcellulose, sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer and polyacrylic acids, preferably xanthan, optionally at least one osmotically active additive and at least one pharmaceutically customary excipient, and the osmosis layer comprises 40% by weight to 90% by weight of at least one hydrophilic swellable polymer preferably selected from a list consisting of xanthan, cellulose derivatives, for example hydroxypropylcellulose, hydroxypropyl methylcellulose, sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer and polyacrylic acids, preferably xanthan and vinylpyrrolidone-vinyl acetate copolymer and optionally at least one pharmaceutically customary excipient.

In a further embodiment, the osmotic release system consists of one of the osmotic release systems described above, wherein the shell consists of cellulose acetate or a mixture of cellulose acetate and polyethylene glycol.

In a further embodiment, the osmotic release system consists of one of the osmotic release systems described above, wherein 80% of the compound of the formula (I) is released after 2 hours to 24 hours, preferably 4 hours to 20 hours, more preferably 5 hours to 16 hours (measured according to the USP dissolution method (USP 39; Chapter <711> Dissolution) using Apparatus 2 (paddle) and the instructions under “Dissolution profile”).

In the context of the present invention, hydrophilic swellable polymers are all pharmaceutically acceptable polymer compounds known to those skilled in the art that swell by taking up water, with the exception of the hydrophilic swellable polymer polyethylene oxide. It is preferable to use at least one hydrophilic swellable polymer selected from a list consisting of xanthan, cellulose derivatives, for example hydroxypropylcellulose, hydroxypropyl methylcellulose or sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids or selected from a list consisting of xanthan, cellulose derivatives, for example hydroxypropylcellulose, hydroxypropyl methylcellulose or sodium carboxymethylcellulose, starch derivatives, for example sodium carboxymethyl starch, vinylpyrrolidone-vinyl acetate copolymer, polyvinylpyrrolidone and methacrylic acid copolymers, for example methacrylic acid-methyl methacrylate copolymer.

It is further preferable to use at least one hydrophilic swellable polymer selected from a list consisting of xanthan, hydroxypropylcellulose, hydroxypropyl methylcellulose, sodium carboxymethylcellulose, sodium carboxymethyl starch, vinylpyrrolidone-vinyl acetate copolymer and polyacrylic acids or selected from a list consisting of xanthan, hydroxypropylcellulose, hydroxypropyl methylcellulose, sodium carboxymethylcellulose, sodium carboxymethyl starch and vinylpyrrolidone-vinyl acetate copolymer, and particularly preferable to use xanthan and vinylpyrrolidone-vinyl acetate copolymer or mixtures thereof.

It is further preferable to use at least one hydrophilic swellable polymer selected from a list consisting of xanthan, Kollidon VA 64, PVP 25, Eudragit L100, Eudragit RL PO, HPC LM and polyacrylic acid or selected from a list consisting of xanthan, Kollidon VA 64, PVP 25, Eudragit L100, Eudragit RL PO and HPC LM.

Starch derivatives suitable as hydrophilic swellable polymers in the context of the present invention are maize, wheat, rice and potato starches, substituted starches such as carboxymethyl starch and the salt thereof, hydroxyethyl starch or mixtures thereof.

Cellulose derivatives suitable as hydrophilic swellable polymers in the context of the present invention are methylcellulose (MC), hydroxymethyl propylcellulose (HPMC), hydroxypropylcellulose (HPC), carboxymethylcellulose sodium (Na-CMC), hydroxyethylcellulose (HEC) or mixtures thereof.

The recited hydrophilic swellable polymers may be used alone or in combination with further hydrophilic swellable polymers.

Some hydrophilic swellable polymers may alternatively be used as pharmaceutically customary excipients in the core, for example as binders or disintegrants. If the proportion of any such substance in the core is equal to or greater than ten percent based on the mass of the core, said substance is regarded as a hydrophilic swellable polymer in the context of the present invention.

Osmotically active additives in the context of the present invention are, for example, all water-soluble substances acceptable for use in pharmaceutics, such as the water-soluble excipients mentioned in pharmacopoeias, in “Hager” and “Remington Pharmaceutical Science” or in other literature (Sareen. R., Jain, N., Kumar, D., Current Drug Delivery, 9, (2012), 285-296). It is possible in particular to use water-soluble salts of inorganic or organic acids or nonionic organic substances with high solubility in water, such as carbohydrates, especially sugars, sugar alcohols or amino acids. For example, the osmotically active additives may be selected from inorganic salts such as chlorides, sulfates, carbonates and bicarbonates of alkali metals or alkaline earth metals such as lithium, sodium, potassium, magnesium and calcium and the phosphates, hydrogen phosphates or dihydrogen phosphates, acetates, succinates, benzoates, citrates or ascorbates thereof. It is further possible to use pentoses such as arabinose, ribose or xylose, hexoses such as glucose, fructose, galactose or mannose, disaccharides such as sucrose, maltose or lactose or trisaccharides such as raffinose. Water-soluble amino acids include glycine, leucine, alanine or methionine. Preference is given to using sodium chloride.

Pharmaceutically customary excipients in the context of the present invention are, for example, buffers such as sodium bicarbonate, binders such as hydroxypropylcellulose, hydroxypropyl methylcellulose, polyvinylpyrrolidone or vinylpyrrolidone-vinyl acetate copolymers (Kollidon® VA64), disintegrants such as sodium carboxymethyl starch, lubricants such as magnesium stearate, wetting agents such as sodium laurylsulfate, flow regulators such as colloidal silica, protective colloids as described in EP-B-0277092 (p. 5, lines 10-25), plasticizers as described for example in EP-B-0277092 (p. 5, lines 29-32), surfactants as described for example in EP-B-0277092 (p. 5, lines 33-44), carrier materials as described for example in EP-B-0277092 (p. 5, lines 45-47), and also one or more colour pigments such as iron oxide in one of the two layers for differentiation between active ingredient layer and osmosis layer. Suitable protective colloids are, for example, methylated cellulose derivatives, e.g. methylcellulose having a methoxy content of about 27.0 to 32.0% and a degree of substitution of about 1.75 to 2.1 or hydroxypropyl methylcellulose having a content of approx. 16.0-30.0% methoxy groups and 4.0-32.0% hydroxypropoxy groups. Suitable plasticizers are, for example, glycerol, triethyl citrate, diethyl phthalate or diethyl sebacate. Suitable surfactants are, for example, anionic surfactants of the alkyl sulfate type, for example sodium, potassium or magnesium n-dodecyl sulfate, n-tetradecyl sulfate, n-hexadecyl sulfate or n-octadecyl sulfate, of the alkyl ether sulfate type, for example sodium, potassium or magnesium n-dodecyloxyethyl sulfate, n-tetradecyloxyethyl sulfate, n-hexadecyloxyethyl sulfate or n-octadecyloxyethyl sulfate or of the alkanesulfonate type, for example sodium, potassium or magnesium n-dodecanesulfonate, n-tetradecanesulfonate, n-hexadecanesulfonate or n-octadecanesulfonate. Suitable surfactants are additionally nonionic surfactants of the fatty acid polyhydroxy alcohol ester type, such as sorbitan monolaurate, monooleate, monostearate or monopalmitate, sorbitan tristearate or trioleate, polyoxyethylene adducts of fatty acid polyhydroxy alcohol esters such as polyoxyethylene sorbitan monolaurate, monooleate, monostearate, monopalmitate, tristearate or trioleate, polyethylene glycol fatty acid esters such as polyoxyethyl stearate, polyethylene glycol 400 stearate, polyethylene glycol 2000 stearate, in particular ethylene oxide-propylene oxide block polymers of the Pluronics® (BWC) or Synperonic® (ICI) type. Suitable carrier materials are, for example, lactose, sucrose, sorbitol, mannitol, starch, for example potato starch, maize starch or amylopectin, or cellulose.

Both in the single-chamber system and in the two-chamber system, the shell of the osmotic active ingredient release system consists of a water-permeable film-forming material that is impermeable to the components of the core. Such shell materials are known in principle and are described for example in EP1024793. Shell materials that may be used are, for example, acylated cellulose derivatives.

Acylated cellulose derivatives (cellulose esters) are celluloses that are mono- to trisubstituted by acetyl groups or mono- to disubstituted by acetyl groups and substituted by a further acyl radical different from acetyl, for example cellulose acetate, cellulose triacetate, cellulose acetate ethylcarbamate, cellulose acetate phthalate, cellulose acetate methylcarbamate, cellulose acetate succinate, cellulose acetate dimethylaminoacetate, cellulose acetate ethylcarbonate, cellulose acetate chloroacetate, cellulose acetate ethyloxalate, cellulose acetate methylsulfonate, cellulose acetate butylsulfonate, cellulose acetate propionate, cellulose acetate diethylaminoacetate, cellulose acetoacetate, cellulose acetate laurate, cellulose acetate p-toluenesulfonate, cellulose acetate butyrate, and shell materials from the cellulose ethers group such as ethylcellulose or other cellulose acetate derivatives and also agar acetate and amylose acetate.

Also suitable as shell materials are ethylcellulose and polymeric epoxides, copolymers of alkylene oxide and alkyl glycidyl ethers, polyglycols, polylactic acid derivatives and other derivatives thereof. In addition, it is also possible to use mixtures of acrylates that are water-insoluble per se (e.g. a copolymer of ethyl acrylate and methyl methacrylate).

In the context of the present invention, preference is given to using cellulose acetate or mixtures of cellulose acetate and polyethylene glycol as the shell material.

The amounts and the constituents used for producing the shell of the osmotic drug release system influence the ingress rate of the gastrointestinal fluid in a known manner. In principle, the ingress rate of the gastrointestinal fluid decreases as the amount of shell material increases.

If required, an outer coating, for example a light-protective coating and/or coloured outer coating may be applied to the shell. Particularly suitable materials are, for example, polymers such as polyvinyl alcohol, hydroxypropylcellulose and/or hydroxypropyl methylcellulose, optionally in combination with suitable plasticizers such as polyethylene glycol or polypropylene glycol, and pigments such as titanium dioxide or iron oxides. One such example is coating with a film coat obtained by initially dissolving polyvinyl alcohol and polyethylene glycol 3350 in water at room temperature and mixing with stirring. Talc, titanium dioxide and iron oxide are added in stages, with stirring. Coating suspensions can for example be applied to the tablet cores using a suitable coating unit, e.g. a Glatt coater. Alternatively, instead of standard coating, sugar coating may be carried out. Such outer coatings are generally applied using an aqueous or organic application medium. In the context of the present invention, the term “outer coating” refers additionally to coatings of the shell applied by an alternative process, for example a solvent-free process.

The coatings used may also be “preformulated” coatings. These already comprise a mixture of excipients and are dissolved in water and applied. An example is Opadry II 85F230009 Orange (Colorcon PVA-based preformulated coating), which comprises partially hydrolysed polyvinyl alcohol, talc, polyethylene glycol (PEG 3350), titanium dioxide, red iron oxide, yellow iron oxide and polysorbate 80 (Tween 80).

The shell of the osmotic drug release system of the present invention has at least one orifice or passage through which the active ingredient together with the other core constituents slowly escapes. The orifice is introduced into the shell by laser drilling, mechanical drilling or e.g. by punching There may be one or more orifices present in the shell. The size of the orifice (diameter) is preferably 0.2 to 1.6 mm, more preferably 0.3 to 1.2 mm. The nature of the orifice and methods for the production thereof are known per se and are described for example in U.S. Pat. Nos. 4,063,064, 4,088,864, 3,916,899 or EP-B-0277092. The optionally present outer coating may likewise have one or more orifices.

As the osmotically active additive in the described embodiments, preference is given to using at least one water-soluble salt of inorganic or organic acids, more preferably sodium chloride.

As pharmaceutically customary excipients in the described embodiments, preference is given to using binders, for example hydroxypropylcellulose, lubricants, for example magnesium stearate, flow regulators, for example colloidal silica, and colour pigments, for example iron oxide.

The osmotic two-chamber system may be produced, for example, by mixing the components of the active ingredient layer and subjecting them to wet or dry, preferably dry, granulation, mixing and granulating the components of the osmosis layer, and then pressing both granulates on a bilayer tablet press to obtain a bilayer tablet. The resulting inner core is then coated with a shell. The shell is, on the active ingredient side, provided with one or more orifices. Alternatively, the introduction of the one or more orifices in this process step may be omitted. In this case, it is only after coating with one or more further outer coatings has been carried out that both sides of the tablet are each provided with an orifice extending in each case from the outside as far as the inner core, i.e. traversing the outer coating and the shell.

In the production of the osmotic two-chamber system, the components of the active ingredient layer and the components of the osmosis layer preferably both undergo granulation, particularly by means of roller granulation.

The dosage forms of the invention have valuable pharmacological properties and can be used for the treatment and/or prevention of disorders in humans and animals.

For the purposes of the present invention, the term “treatment” or “treating” includes inhibition, retardation, halting, alleviating, attenuating, restricting, reducing, suppressing, reversing or healing of a disease, a condition, a disorder, an injury or a health problem or of the development, course or progression of such states and/or symptoms of such states. The term “therapy” is understood here to be synonymous with the term “treatment”.

The terms “prevention” and “prophylaxis” are used synonymously in the context of the present invention and refer to the avoidance or reduction of the risk of contracting, experiencing, suffering from or having a disease, a condition, a disorder, an injury or a health problem or a development or progression of such states and/or the symptoms of such states.

The treatment or prevention of a disease, a condition, a disorder, an injury or a health problem may be partial or complete.

The dosage forms of the invention result in vascular relaxation, inhibition of platelet aggregation and lowering of blood pressure and also boost coronary blood flow and the microcirculation. These effects are mediated by a direct, haem-independent activation of soluble guanylate cyclase and a rise in intracellular cGMP levels.

The dosage forms of the invention are especially suitable for the treatment and/or prevention of renal and cardiorenal disorders, in particular chronic kidney disease (CKD) and diabetic kidney disease (DKD), cardiac and cardiovascular disorders, in particular heart failure (HFpEF and HFrEF), myocardial infarction, angina pectoris, cardiomyopathies, hypertension and arteriosclerosis, pulmonary and cardiopulmonary disorders, in particular pulmonary hypertension (PH), disorders of the central nervous system, in particular dementia, bone disorders, in particular osteogenesis imperfecta, thromboembolic disorders, muscular dystrophies, ischaemias, vascular disorders, microcirculation impairment, fibrotic disorders, in particular systemic sclerosis, in particular age-related macular degeneration, inflammatory disorders, and metabolic disorders, in particular metabolic syndrome, dyslipidaemia and diabetes.

The dosage forms of the invention can be used for the treatment and/or prevention of cardiac, cardiovascular and cardiopulmonary disorders, for example high blood pressure (hypertension), heart failure, coronary heart disease, stable and unstable angina pectoris, pulmonary arterial hypertension (PAH) and secondary forms of pulmonary hypertension (PH), chronic thromboembolic pulmonary hypertension (CTEPH), renal hypertension, disorders of peripheral and cardiac vessels, arrhythmias, atrial and ventricular arrhythmias and impaired conduction, for example grade I-III atrioventricular block, supraventricular tachyarrhythmia, atrial fibrillation, atrial flutter, ventricular fibrillation, ventricular flutter, ventricular tachyarrhythmia, torsade-de-pointes tachycardia, atrial and ventricular extrasystoles, AV junctional extrasystoles, sick sinus syndrome, syncope, AV node reentry tachycardia, Wolff-Parkinson-White syndrome, acute coronary syndrome (ACS), autoimmune heart disorders (pericarditis, endocarditis, valvulitis, aortitis, cardiomyopathies), boxer cardiomyopathy, aneurysms, shock such as cardiogenic shock, septic shock and anaphylactic shock.

The dosage forms of the invention can be used for the treatment and/or prevention of thromboembolic disorders and ischaemias such as myocardial ischaemia, myocardial infarction, stroke, cardiac hypertrophy, transitory and ischaemic attacks, preeclampsia, inflammatory cardiovascular disorders, spasms of the coronary arteries and the peripheral arteries, formation of oedemas, for example, pulmonary oedema, cerebral oedema, renal oedema or heart failure-induced oedema, peripheral circulation impairment, reperfusion damage, arterial and venous thrombosis, microalbuminuria, myocardial insufficiency, endothelial dysfunction, micro- and macrovascular damage (vasculitis), and also for preventing restenosis for example after thrombolysis therapies, percutaneous transluminal angioplasties (PTA), percutaneous transluminal coronary angioplasties (PTCA), heart transplants, bypass operations and micro- and macrovascular damage (vasculitis), elevated levels of fibrinogen and of low-density LDL and elevated concentrations of plasminogen activator inhibitor 1 (PAI-1), and for the treatment and/or prophylaxis of erectile dysfunction and female sexual dysfunction.

For the purposes of the present invention, the term “pulmonary hypertension” encompasses both primary and secondary subforms thereof as defined by the Dana Point classification in accordance with the aetiology in the individual case [see D. Montana and G. Simonneau, in: A. J. Peacock et al. (eds.), Pulmonary Circulation. Diseases and their treatment, 3rd edition, Hodder Arnold Publ., 2011, pp. 197-206; M. M. Hoeper et al., J. Am. Coll. Cardiol., 2009, 54 (1), pp. 85-96]. This includes, in group 1 in particular, pulmonary arterial hypertension (PAH), which includes inter alia the idiopathic and familial forms (IPAH and FPAH, respectively), acute pulmonary hypertension, in particular acute respiratory distress syndrome (ARDS), acute lung injury (ALI) and infant respiratory distress syndrome (IRDS). PAH also encompasses persistent pulmonary hypertension of the newborn and associated pulmonary arterial hypertension (APAH), which is associated with collagenoses, congenital systemic-to-pulmonary shunts, portal hypertension, HIV infection, the use of certain drugs and medicaments (for example of appetite suppressants), with disorders having a significant venous/capillary component such as pulmonary venoocclusive disorder and pulmonary capillary haemangiomatosis, or with other disorders such as thyroid disorders, glycogen storage diseases, Gaucher's disease, hereditary telangiectasia, haemoglobinopathies, myeloproliferative disorders and splenectomy. Group 2 of the Dana Point classification covers PH patients with disorders that are left-ventricular in origin, such as ventricular, atrial or valvular disorders. Group 3 includes forms of pulmonary hypertension associated with lung disease, for example chronic obstructive lung disease (COPD), interstitial lung disease (ILD), pulmonary fibrosis (IPF), and/or hypoxaemia, sleep apnoea, alveolar hypoventilation, chronic altitude sickness, constitutional deformities. Group 4 includes PH patients with chronic thrombotic and/or embolic disorders, for example in thromboembolic obstruction of proximal and distal pulmonary arteries (CTEPH) or in non-thrombotic embolisms (e.g. as a result of tumour diseases, parasites, foreign bodies). Less common forms of pulmonary hypertension, such as in patients with sarcoidosis, Langerhans cell histiocytosis or lymphangiomatosis, are covered by group 5.

For the purposes of the present invention, the term “heart failure” encompasses both acute and chronic forms of heart failure and also specific or related disease types thereof, such as acute decompensated heart failure, right-ventricular failure, left-ventricular failure, global failure, ischaemic cardiomyopathy, dilatative cardiomyopathy, hypertrophic cardiomyopathy, idiopathic cardiomyopathy, congenital heart defects, valvular heart disease, heart failure associated with valvular heart disease, mitral stenosis, mitral insufficiency, aortic stenosis, aortic insufficiency, tricuspid stenosis, tricuspid insufficiency, pulmonary stenosis, pulmonary insufficiency, combined valvular heart disease, myocardial inflammation (myocarditis), chronic myocarditis, acute myocarditis, viral myocarditis, diabetic heart failure, alcoholic cardiomyopathy, cardiac storage disorders and diastolic and systolic heart failure, heart failure with reduced ejection fraction (HFrEF), heart failure with preserved ejection fraction (HFpEF).

The dosage forms of the invention are also suitable for the treatment and/or prevention of metabolic disorders. In the context of the present invention, metabolic disorders are, for example, disorders of glucose metabolism and disorders and complications associated with impaired glucose metabolism. Disorders of glucose metabolism are, for example, diabetes mellitus (type 1 or type 2), insulin resistance, impaired glucose tolerance, hyperglycaemia, hypoglycaemia, hyperinsulinaemia or hypoinsulinaemia. Disorders associated with impaired glucose metabolism are, for example, micro- and macroangiopathies, diabetic retinopathies, diabetic neuropathies, diabetic nephropathies, delayed/impaired wound healing, diabetic foot, tissue ischaemias, ulcers on the extremities, gangrene, metabolic acidosis, ketosis, dyslipidaemias, myocardial infarction, acute coronary syndrome, stable or unstable angina pectoris, cardiomyopathies, heart failure, cardiac arrhythmias, vascular restenosis, peripheral arterial occlusive disease, obesity, syndrome X, impaired fat metabolism, arteriosclerosis or high blood pressure. The compound of the formula (I) according to the invention and the dosage forms of the invention are also suitable for maintaining, improving and restoring the functions of pancreatic cells, in particular for maintaining, improving and restoring the number and size of pancreatic beta cells.

In the context of the present invention, metabolic disorders also include disorders of fat metabolism such as impaired lipid metabolism, hypolipoproteinaemias, dyslipidaemias, hypertriglyceridaemias, hyperlipidaemias, combined hyperlipidaemias, hypercholesterolaemias, abetalipoproteinaemia, sitosterolaemia, xanthomatosis, Tangier disease, adiposity, obesity, arteriosclerosis and metabolic syndrome. The dosage forms of the invention are also suitable for the treatment and/or prevention of cardiovascular disorders associated with a metabolic disorder.

The dosage forms of the invention are also suitable for the treatment and/or prevention of muscular or neuromuscular disorders. The expression “muscular or neuromuscular disorders” refers to a medical condition affecting the muscles and/or their direct control of the nervous system. They may be acquired or of genetic origin. Muscular or neuromuscular disorders are in particular Duchenne muscular dystrophy (DMD), Becker muscular dystrophy (BMD), congenital muscular dystrophy, Miyoshi myopathy, Emery-Dreifuss muscular dystrophy, facioscapulohumeral muscular dystrophy, limb-girdle muscular dystrophy, myotonic muscular dystrophy, oculopharyngeal muscular dystrophy, myasthenia gravis, Lambert-Eaton myasthenic syndrome and Charcot-Marie-Tooth disease.

In addition, the dosage forms of the invention can be used for the treatment and/or prevention of primary and secondary Raynaud phenomena, microcirculation impairment, claudication, hearing impairment, tinnitus, peripheral and autonomous neuropathies, diabetic microangiopathies, diabetic retinopathy, CREST syndrome, erythematosis, onychomycosis and rheumatic disorders.

The dosage forms of the invention can also be used for the treatment and/or prevention of ischaemia- and/or reperfusion-related damage to organs or tissues and as additives for perfusion and preservation solutions for organs, organ parts, tissues or tissue parts of human or animal origin, particularly in surgical interventions or in the field of transplantation medicine.

The dosage forms of the invention are also suitable for the treatment and/or prevention of renal disorders, in particular renal insufficiency and kidney failure. For the purposes of the present invention, the terms “renal insufficiency” and “kidney failure” encompass both acute and chronic manifestations thereof (chronic kidney disease; CKD) and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and immune complex-induced kidney disorders, nephropathy induced by toxic substances, nephropathy induced by contrast agents, diabetic and non-diabetic nephropathy, diabetic kidney disease (DKD), pyelonephritis, renal cysts, nephrosclerosis, hypertensive nephrosclerosis and nephrotic syndrome, which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomeruli and arterioles, tubular dilatation, hyperphosphataemia and/or need for dialysis. The present invention also encompasses the use of the dosage forms of the invention for the treatment and/or prevention of sequelae of renal insufficiency, for example hypertension, pulmonary oedema, heart failure, uraemia, anaemia, electrolyte disturbances (for example hyperkalaemia, hyponatraemia) and disturbances of bone and carbohydrate metabolism.

In addition, the dosage forms of the invention are suitable for the treatment and/or prevention of disorders of the urogenital system, for example benign prostatic syndrome (BPS), benign prostatic hyperplasia (BPH), benign prostatic enlargement (BPE), bladder outlet obstruction (BOO), lower urinary tract syndromes (LUTS), interstitial cystitis, neurogenic overactive bladder (OAB), incontinence, for example mixed urinary incontinence (MUI), urge urinary incontinence (UUI), stress urinary incontinence (SUI) or overflow urinary incontinence (OUI), pelvic pain, and also erectile dysfunction, female sexual dysfunction, vaginal atrophy, dyspareunia or atrophic vaginitis.

The dosage forms of the invention are also suitable for the treatment and/or prevention of asthmatic disorders, chronic-obstructive pulmonary diseases (COPD), acute respiratory distress syndrome (ARDS) and acute lung injury (ALI), alpha-1 antitrypsin deficiency (AATD), pulmonary fibrosis, pulmonary emphysema (for example pulmonary emphysema induced by cigarette smoke), pulmonary venous hypertension, interstitial lung disease, sleep apnoea, alveolar hypoventilation disorders, chronic exposure to high altitudes, neonatal lung disease, alveolar capillary dysplasia, sickle cell anaemia, impaired coagulation, chronic thromboembolism, tumour-associated pulmonary embolism, connective-tissue disorders, lupus, schistosomiasis, sarcoidosis, chronic bronchitis, capillary pulmonary haemangiomatosis; Langerhans cell histiocytosis, lymphangiomatosis and compression of the pulmonary vessels secondary to adenopathy, fibrosing mediastinitis and cystic fibrosis (CF).

The dosage forms of the invention described in the present invention also constitute dosage forms for the control of central nervous system disorders characterized by disturbances of the NO/cGMP system. They are particularly suitable for improving perception, concentration, learning or memory after cognitive impairment such as occur especially in situations/diseases/syndromes such as mild cognitive impairment, age-associated learning and memory impairment, age-associated memory loss, dementia, vascular dementia, mixed forms of dementia, post-stroke dementia, post-traumatic brain injury, general concentration impairment, concentration impairment in children with learning and memory problems, Alzheimer's dementia, Lewy body dementia, dementia with frontal-lobe degeneration including Pick's syndrome, Parkinson's disease, progressive nuclear palsy, dementia with corticobasal degeneration, amyolateral sclerosis (ALS), Huntington's disease, demyelination, multiple sclerosis, thalamic degeneration, Creutzfeldt-Jacob dementia, HIV dementia, schizophrenia with dementia or Korsakoff s psychosis, Binswanger dementia (subcortical arteriosclerotic encephalopathy), cerebral autosomal-dominant arteriopathy with subcortical infarcts and leukoencephalopathy (iCADASIL or CADASIL syndrome), asymptomatic neurocognitive impairment (ANI), multiple sclerosis (MS) (including clinically isolated syndrome (CIS), relapsing-remitting MS (RRMS), primary progressive MS (PPMS) and secondary progressive MS (SPMS), multisystem atrophy (MSA), Parkinson's disease, Parkinson's plus, progressive supranuclear palsy (PSP, Steele-Richardson-Olszewski syndrome), attention deficit syndrome (ADS) and attention deficit-hyperactivity disorder (ADHS). They are also suitable for the treatment and/or prevention of central nervous system disorders such as states of anxiety, tension and depression, CNS-related sexual dysfunction and sleep disturbances, and for controlling pathological disturbances of the intake of food, stimulants and addictive substances. They are also suitable for the treatment and/or prevention of injuries, for example traumatic brain injury (TBI) including, for example, concussion and traumatic encephalopathies (CTE), or non-traumatic strokes (including ischaemic strokes, aneurysms or hypoxias), brain damage, cognitive impairment, brain injuries, neurodegenerative disorders or neuropathic pain. They are also suitable for the treatment and/or prevention of dystonias, for example generalized, focal, segmental, vegetative, acute dystonic reactions and genetic/primary dystonias and dyskinesias, including acute, chronic/tardive and non-motor and levodopa-induced dyskinesias (LID). They are also suitable for the treatment and/or prevention of disorders characterized by a reduction in synaptic plasticity and in synaptic processes, for example fragile X syndrome, Rett syndrome, Williams syndrome, Renpenning's syndrome, autistic spectrum disorders including autism, Asperger's syndrome or pervasive development disorders. They are also suitable for the treatment and/or prevention of mental, affective or psychological disorders, for example bipolar disorder, schizophrenia, general psychosis, drug-induced psychosis, paranoia, schizoaffective disorder, obsessive-compulsive disorder (OCD), depressive disorders, anxiety disorders, panic disorders or post-traumatic stress disorder (PTSD).

In addition, the dosage forms of the invention are also suitable for the regulation of cerebral blood flow and are thus effective agents for controlling migraine. They are also suitable for the prophylaxis and control of sequelae of cerebral infarct events (apoplexia cerebri) such as stroke, cerebral ischaemias and traumatic brain injury. The dosage forms of the invention can likewise be used for controlling states of pain.

In addition, the dosage forms of the invention have anti-inflammatory effects and can therefore be used as anti-inflammatory agents for the treatment and/or prevention of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders and inflammatory skin disorders.

The dosage forms of the invention are also suitable for the treatment and/or prevention of acute pain, central pain syndrome, chemotherapy-induced neuropathy and neuropathic pain, diabetic neuropathy, fibromyalgia, inflammatory pain, neuropathic pain, postoperative pain, tonic pain or visceral pain.

The dosage forms of the invention are also suitable for the treatment and/or prevention of fibrotic disorders of the internal organs, for example the lung, heart, kidneys, bone marrow and especially the liver, and also dermatological fibroses and fibrotic eye disorders. For the purposes of the present invention, the term “fibrotic disorders” encompasses in particular disorders such as hepatic fibrosis, cirrhosis of the liver, pulmonary fibrosis, endomyocardial fibrosis, nephropathy, glomerulonephritis, interstitial renal fibrosis, fibrotic damage secondary to diabetes, bone marrow fibrosis and similar fibrotic disorders, scleroderma, systemic sclerosis, morphea, keloids, hypertrophic scarring, naevi, diabetic retinopathy, proliferative vitreoretinopathy and connective tissue disorders (for example sarcoidosis). The dosage forms of the invention can likewise be used for treating steatohepatitis, in particular non-alcoholic steatohepatitis (NASH), for promoting wound healing, for controlling postoperative scarring, for example after glaucoma operations and for cosmetic purposes in ageing and keratinized skin.

In addition, the dosage forms of the invention are suitable for the treatment and/or prevention of bone disorders, for example and preferably osteogenesis imperfecta (OI), bone fractures, impaired bone healing, rickets, osteomalacia, avascular bone necrosis, Paget's disease, osteodystrophy, osteopenia, osteolytic lesions caused by bone metastases, radiation therapy or chemotherapy, parodontitis, hypercalcaemia, osteonecrosis, osteosarcoma, osteolytic metastases, familial expansile osteolysis, expansile skeletal and idiopathic hyperplasia, juvenile Paget's disease, Camurati-Engelmann disease, loosening of prostheses, periprosthetic osteolysis, cleidocranial dysplasia (CCD), multiple myeloma, alveolar bone loss, bone loss caused by immobilization or sex hormone deficiency, bone loss associated with a disease selected from the group consisting of cachexia, anorexia, alopecia and inflammatory disorders selected from the group consisting of rheumatoid arthritis, psoriatic arthritis, psoriasis, spondyloarthritis, SLE, systemic sclerosis, metastatic cancer and inflammatory bowel disease, osteoarthritis, impaired bone healing after osteotomy, idiopathic bone loss in infancy, curvature of the spine, osteoporosis, primary osteoporosis, secondary osteoporosis and especially osteoporosis, primary osteoporosis or secondary osteoporosis not caused by sex hormone deficiency.

In addition, the dosage forms of the invention are suitable for the treatment and/or prevention of dysfunctions of gastrointestinal sphincters, such as achalasia, sphincter spasms and hypertensive sphincter, especially lower oesophagus sphincter (LES) achalasia, oesophagus achalasia, spastic LES, hypertension of the LES (HTNLES), pyloric sphincter (pyloric) achalasia, pyloric spasm (pylorospasm), pyloric hypertension, ileocaecal sphincter or valve (ICV) achalasia, hypertensive ICV, spastic ICV or ICV spasm, sphincter of Oddi dysfunction (SOD), sphincter of Oddi achalasia, spastic sphincter of Oddi, sphincter of Oddi hypertension, internal anal sphincter (IAS) achalasia, IAS hypertension, spastic IAS or IAS spasm. In a further embodiment, the gastrointestinal sphincter dysfunctions mentioned are attributable to a neurological, metabolic, endocrine or neurodegenerative disorder.

In addition, the dosage forms of the invention are suitable for the treatment and/or prevention of ophthalmological disorders that for the purposes of the invention should be understood as meaning, for example, the following disorders: age-related macular degeneration (AMD) including dry (non-exudative) and wet (exudative, neovascular) AMD, choroidal neovascularization (CNV), choroidal neovascular membranes (CNVM), cystoid macular oedema (CME), epiretinal membranes (ERM) and macular perforations, myopia-associated choroidal neovascularization, angioid and vascular streaks, retinal detachment, diabetic retinopathy, non-proliferative diabetic retinopathy (NPDR), diabetic macular oedema (DMO), atrophic and hypertrophic changes to the retinal pigment epithelium, retinal vein occlusion, choroidal retinal vein occlusion, macular oedema, macular oedema associated with retinal vein occlusion, retinitis pigmentosa, Stargardt disease, retinopathy of prematurity, glaucoma, inflammatory disorders of the eye, for example uveitis, scleritis or endophthalmitis, cataract, refractive anomalies, for example myopia, hyperopia, astigmatism or keratoconus, corneal angiogenesis secondary to e.g. keratitis, corneal transplant or keratoplasty, corneal angiogenesis secondary to hypoxia (for example due to extensive wearing of contact lenses), pterygium conjunctivae, subcorneal oedema and intracorneal oedema.

The activity profile of the dosage forms of the invention makes them particularly suitable for the treatment and/or prevention of cardiovascular and cardiopulmonary disorders such as primary and secondary forms of pulmonary hypertension, heart failure, angina pectoris and hypertension and also of thromboembolic disorders, ischaemias, vascular disorders, microcirculation impairment, renal insufficiency, fibrotic disorders and arteriosclerosis.

The dosage forms of the invention are preferably suitable for the treatment and/or prevention of renal and cardiorenal disorders, in particular chronic kidney disease (CKD) and diabetic kidney disease (DKD), cardiac and cardiovascular disorders, in particular heart failure (HFpEF and HFrEF), myocardial infarction, angina pectoris, cardiomyopathies, hypertension and arteriosclerosis, pulmonary and cardiopulmonary disorders, in particular pulmonary hypertension (PH), ophthalmological disorders, in particular non-proliferative diabetic retinopathy (NPDR) and diabetic macular oedema (DMO), disorders of the central nervous system, in particular dementia, bone disorders, in particular osteogenesis imperfecta, thromboembolic disorders, muscular dystrophies, ischaemias, vascular disorders, microcirculation impairment, fibrotic disorders, in particular systemic sclerosis, inflammatory disorders, and metabolic disorders, in particular metabolic syndrome, dyslipidaemia and diabetes.

The dosage forms of the invention are particularly suitable for the treatment and/or prevention of renal and cardiorenal diseases, in particular chronic kidney disease (CKD).

The dosage forms of the invention are particularly suitable for the treatment and/or prevention of ophthalmological disorders, in particular non-proliferative diabetic retinopathy (NPDR) and diabetic macular oedema (DMO).

The dosage forms of the invention are particularly suitable for the treatment and/or prevention of cardiovascular disorders, in particular heart failure, including heart failure with reduced ejection fraction (HFrEF) and heart failure with preserved ejection fraction (HFpEF).

The dosage forms of the invention are particularly suitable for the treatment and/or prevention of cardiopulmonary disorders, in particular pulmonary hypertension.

The dosage forms of the invention are particularly suitable for the treatment and/or prevention of disorders of the central nervous system, in particular dementia, including vascular dementia and mixed forms of dementia.

The dosage forms of the invention are particularly suitable for the treatment and/or prevention of “muscular or neuromuscular disorders”, in particular Duchenne muscular dystrophy (DMD) and Becker muscular dystrophy (BMD).

The present invention further provides for the use of the dosage forms of the invention for the treatment and/or prevention of sickle cell anaemia, wherein traumatized patients receive a synthetic blood substitute, and for the preservation of blood substitutes.

The present invention further provides for the use of the dosage forms of the invention for the treatment and/or prevention of polycystic ovary syndrome (PCOS).

The present invention further provides for the use of the dosage forms of the invention for the treatment and/or prevention of preeclampsia.

The present invention further provides for the use of the dosage forms of the invention for the treatment and/or prevention of disorders, especially of the abovementioned disorders.

The present invention further provides for the use of the dosage forms of the invention in a method for the treatment and/or prevention of disorders, especially of the abovementioned disorders.

The present invention further provides a method for the treatment and/or prevention of disorders, especially of the abovementioned disorders, using at least one of the dosage forms of the invention.

The dosage forms of the invention may be used alone or, if required, in combination with other active substances. The present invention further provides medicaments comprising at least one of the dosage forms of the invention and one or more further active substances, especially for the treatment and/or prophylaxis of the abovementioned disorders. Preferred examples of active substances suitable for combinations include:
organic nitrates and NO donors, for example sodium nitroprusside, glyceryl trinitrate, isosorbide mononitrate, isosorbide dinitrate, molsidomine or SIN-1, and inhaled NO;
further substances that increase the cGMP concentration, for example protoporphyrin IX, arachidonic acid or phenylhydrazine derivatives;
NO synthase substrates, for example N-hydroxyguanidine derivatives, L-arginine derivatives, N-alkyl-N′-hydroxyguanidine derivatives, N-aryl-N′-hydroxyguanidine derivatives or guanidine derivatives;
compounds that inhibit the breakdown of cyclic guanosine monophosphate (cGMP) and/or cyclic adenosine monophosphate (cAMP), for example inhibitors of phosphodiesterases (PDE) 1, 2, 3, 4, 5, 9 and/or 10, especially PDE 4 inhibitors such as roflumilast or revamilast and PDE 5 inhibitors such as sildenafil, vardenafil, tadalafil, udenafil, dasantafil, avanafil, mirodenafil or lodenafil;
NO-independent but haem-dependent stimulators of guanylate cyclase, especially riociguat, nelociguat, vericiguat, praliciguat (IW-1973), olinciguat (IW-1701) and the compounds described in WO 00/06568, WO 00/06569, WO 02/42301, WO 03/095451, WO 2011/147809, WO 2012/004258, WO 2012/028647 and WO 2012/059549;
prostacyclin analogues and IP receptor agonists, for example and preferably iloprost, beraprost, treprostinil, epoprostenol, NS-304, selexipag or ralinepag;
endothelin receptor antagonists, for example and preferably bosentan, darusentan, ambrisentan, macicentan or sitaxsentan;
inhibitors of human neutrophil elastase (HNE), for example and preferably sivelestat or DX-890 (Reltran);
compounds that inhibit the signal transduction cascade, in particular from the group of tyrosine kinase inhibitors, for example and preferably dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib, masitinib or tandutinib;
Rho kinase inhibitors, for example and preferably fasudil, Y-27632, SLx-2119, BF-66851, BF-66852, BF-66853, KI-23095 or BA-1049;
anti-obstructive agents such as those used for example in the therapy of chronic obstructive pulmonary disease (COPD) or bronchial asthma, for example and preferably inhalatively or systemically administered beta-receptor mimetics (e.g. bedoradrine) or inhalatively administered antimuscarinic substances;
anti-inflammatory and/or immunosuppressive agents such as those used for example in the therapy of chronic obstructive pulmonary disease (COPD), bronchial asthma or pulmonary fibrosis, for example and preferably systemically or inhalatively administered corticosteroids, flutiform, pirfenidone, acetylcysteine, azathioprine or BIBF-1120;
chemotherapeutics such as those used for example in the therapy of neoplasms of the lung or other organs;
active substances used for the systemic and/or inhalative treatment of pulmonary disorders, for example for cystic fibrosis (alpha-1-antitrypsin, aztreonam, ivacaftor, lumacaftor, ataluren, amikacin, levofloxacin), chronic obstructive pulmonary disease (COPD) (LAS40464, PT003, SUN-101), acute respiratory distress syndrome (ARDS) and acute lung injury (ALI) (interferon-beta-1a, traumakines), obstructive sleep apnoea (VI-0521), bronchiectasis (mannitol, ciprofloxacin), bronchiolitis obliterans (ciclosporin, aztreonam) and sepsis (pagibaximab, Voluven, ART-123);
active substances used for the treatment of muscular dystrophy, for example idebenone;
antithrombotic agents, for example and preferably from the group of platelet aggregation inhibitors, anticoagulants or profibrinolytic substances;
active substances that alter lipid metabolism, for example and preferably from the group of thyroid receptor agonists, cholesterol synthesis inhibitors, for example and preferably HMG-CoA reductase inhibitors or squalene synthesis inhibitors, ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein (a) antagonists;
active substances that inhibit neoangiogenesis, for example and preferably inhibitors of the VEGF and/or PDGF signalling pathways, inhibitors of the integrin signalling pathways, inhibitors of the angiopoietin-Tie signalling pathways, inhibitors of the PI3K-Akt-mTor signalling pathways, inhibitors of the Ras-Raf-Mek-Erk signalling pathway, inhibitors of the MAPK signalling pathways, inhibitors of the FGF signalling pathways, inhibitors of the sphingosine-1-phosphate signalling pathways, inhibitors of endothelial cell proliferation or apoptosis-inducing active substances;
active substances that reduce vascular wall permeability (oedema formation), for example and preferably corticosteroids, inhibitors of the ALK1-Smad1/5 signalling pathway, inhibitors of the VEGF and/or PDGF signalling pathways, cyclooxygenase inhibitors, inhibitors of the kallikrein-kinin system or inhibitors of the sphingosine-1-phosphate signalling pathways;
active substances that reduce damage to the retina under oxidative stress, for example and preferably inhibitors of the complement system, especially complement C5a receptor antagonists, or 5-HT_{1A} receptor agonists;
antioxidants and free-radical scavengers;
antihypertensive active substances, for example and preferably from the group of calcium antagonists, angiotensin AII antagonists, ACE inhibitors, beta-receptor blockers, alpha-receptor blockers, diuretics, phosphodiesterase inhibitors, sGC stimulators, cGMP elevating drugs, ECE inhibitors, vasopeptidase inhibitors and/or mineralocorticoid receptor antagonists;
antiarrhythmics, for example sodium-channel blockers, beta-receptor blockers, potassium-channel blockers or calcium-channel blockers;
alpha-1-adrenoceptor antagonists;
centrally acting alpha-2-adrenoceptor agonists;
imidazoline I-1 receptor agonists;
dopamine D1 receptor agonists;
5-HT2 antagonists;
vasopressin antagonists;
calcium channel sensitizers;
bronchodilators, for example beta-2-adrenoceptor agonists, anticholinergics, theophylline or PDE inhibitors;
corticosteroids, for example prednisolone;
PGD2 receptor antagonists;
nonsteroidal antiasthmatics, for example beta-2-adrenoceptor agonists or combinations of beta-2-adrenoceptor agonists and corticosteroids;
nonsteroidal anti-inflammatory drugs (NSAIDs) and selective cyclooxygenase-2 (COX-2) inhibitors;
drugs for excess weight and obesity, for example methamphetamine, amfepramone, phentermine, benzphetamine, phendimetrazine, mazindol, orlistat, sibutramine or rimonabant and combinations, for example phentermine/topiramate, bupropion/naltrexone, sibutramine/metformin, bupropion SR/zonisamide SR, salmeterol, xinafoate/fluticasone;
lorcaserin, phentermine/topiramate, cetilistat, exenatide, liraglutide, metformin, sibutramine/metformin, bupropion SR/zonisamide SR, CORT-108297, canagliflozin, chromium picolinate, GSK-1521498, LY-377604, metreleptin, obinepitide, P-S7AS3, PSN-821, salmeterol xinafoate/fluticasone, somatropin (recombinant), tesamorelin, tesofensine, velneperit, zonisamide, beloranib, resveratrol, sobetirome, tetrahydrocannabivarin and beta-lapachone;
adenylate cyclase inhibitors, for example colforsin dapropate;
positive inotropic substances, for example digoxin;
drugs for the treatment of erectile dysfunction, for example alprostadil;
antidementia drugs such as acetylcholinesterase inhibitors, for example donepezil, galantamine and rivastigmine; or NMDA receptor antagonists, for example memantine;
drugs for the treatment of psychological disorders, for example dopamine D4 receptor antagonists, such as clozapine, dopamine D2 receptor antagonists, such as nemonapride, mixed dopamine D 1/D2 receptor antagonists, such as zuclopenthixol, GABA A receptor modulators, such as carbamazepine, sodium channel inhibitors, such as lamotrigine, monoamine oxidase inhibitors, such as moclobemide, tricyclic antidepressants, such as amitriptyline, desipramine, imipramine, amoxapine, nortriptyline or clomipramine, selective serotonin reuptake inhibitors (SSRIs), such as paroxetine, fluoxetine or citralopram, doxepine, trazodone or agomelatine, selective noradrenaline reuptake inhibitors (SNRls), such as venlafaxine or dopaminergic antidepressants, such as bupropion;
inhibitors of neural endopeptidase (NEP inhibitors) such as sacubitril, omapatrilat or methylene blue, AVE-7688, or in dual combination (“ARMs”) with angiotensin receptor blockers (e.g. valsartan), for example LCZ696;
natriuretic peptides, for example atrial natriuretic peptide (ANP, anaritide), B-type natriuretic peptide or brain natriuretic peptide (BNP, nesiritide), C-type natriuretic peptide (CNP) and urodilatin;
antidiabetics, for example and preferably from the group of the insulins and insulin derivatives, sulfonylureas, biguanides, meglitinide derivatives, glucosidase inhibitors, PPAR-gamma agonists, GLP 1 receptor agonists, glucagon antagonists, insulin sensitizers, CCK1 receptor agonists, dipeptidylpeptidase 4 inhibitors (gliptins), SGLT 2 inhibitors, leptin receptor agonists, potassium channel antagonists and inhibitors of liver enzymes involved in the stimulation of gluconeogenesis and/or glycogenolysis;
antiinfectives, for example and preferably from the group of antibacterial, antifungal and/or antiviral substances; and/or
substances for the treatment of glaucoma, for example and preferably from the group of adrenergics, beta-receptor blockers, carbonic anhydrase inhibitors, parasympathomimetics and prostaglandins; and/or
substances for the treatment of bone disorders, for example and preferably bisphosphonates, vitamin D or metabolites thereof, strontium ranelate, selective oestrogen receptor modulators (SERM), parathyroid hormone or analogues thereof and/or RANKL (receptor activator of nuclear factor kappa-B ligand) modulators.

Antithrombotic agents are preferably understood as meaning compounds from the group of platelet aggregation inhibitors, anticoagulants or profibrinolytic substances.

In a preferred embodiment of the invention, the dosage forms of the invention are administered in combination with a platelet aggregation inhibitor, for example and preferably aspirin, clopidogrel, ticlopidine or dipyridamole.

In a preferred embodiment of the invention, the dosage forms of the invention are administered in combination with a thrombin inhibitor, for example and preferably ximelagatran, melagatran, dabigatran, bivalirudin or clexane.

In a preferred embodiment of the invention, the dosage forms of the invention are administered in combination with a GPIIb/IIIa antagonist, for example and preferably tirofiban or abciximab.

In a preferred embodiment of the invention, the dosage forms of the invention are administered in combination with a factor Xa inhibitor, for example and preferably rivaroxaban, apixaban, fidexaban, razaxaban, fondaparinux, idraparinux, DU-176b, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the dosage forms of the invention are administered in combination with heparin or with a low-molecular-weight (LMW) heparin derivative.

In a preferred embodiment of the invention, the dosage forms of the invention are administered in combination with a vitamin K antagonist, for example and preferably coumarin, phenprocoumon or warfarin.

Antihypertensives are preferably understood as meaning compounds from the group of calcium antagonists, angiotensin AII antagonists, ACE inhibitors, endothelin antagonists, renin inhibitors, alpha-receptor blockers, beta-receptor blockers, mineralocorticoid receptor antagonists, and diuretics.

In a preferred embodiment of the invention, the dosage forms of the invention are administered in combination with a calcium antagonist, for example and preferably nifedipine, amlodipine, verapamil or diltiazem.

In a preferred embodiment of the invention, the dosage forms of the invention are administered in combination with an alpha-1 receptor blocker, for example and preferably prazosin.

In a preferred embodiment of the invention, the dosage forms of the invention are administered in combination with a beta receptor blocker, for example and preferably propranolol, atenolol, timolol, pindolol, alprenolol, oxprenolol, penbutolol, bupranolol, metipranolol, nadolol, mepindolol, carazolol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the dosage forms of the invention are administered in combination with an angiotensin AII antagonist, for example and preferably losartan, candesartan, valsartan, telmisartan or embursatan.

In a preferred embodiment of the invention, the dosage forms of the invention are administered in combination with an ACE inhibitor, for example and preferably enalapril, captopril, lisinopril, ramipril, delapril, fosinopril, quinopril, perindopril or trandolapril.

In a preferred embodiment of the invention, the dosage forms of the invention are administered in combination with an endothelin antagonist, for example and preferably bosentan, darusentan, ambrisentan or sitaxsentan.

In a preferred embodiment of the invention, the dosage forms of the invention are administered in combination with a renin inhibitor, for example and preferably aliskiren, SPP-600 or SPP-800.

The dosage forms of the invention are administered in combination with a mineralocorticoid receptor antagonist, for example spironolactone or eplerenone, particularly preferably with a nonsteroidal mineralocorticoid receptor antagonist such as finerenone.

In a preferred embodiment of the invention, the dosage forms of the invention are administered in combination with a diuretic, for example and preferably furosemide, bumetanide, torasemide, bendroflumethiazide, chlorthiazide, hydrochlorthiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

Modifiers of lipid metabolism are preferably understood as meaning compounds from the group of CETP inhibitors, thyroid receptor agonists, cholesterol synthesis inhibitors such as HMG-CoA reductase inhibitors or squalene synthesis inhibitors, ACAT inhibitors, MTP inhibitors, PPAR-alpha, PPAR-gamma and/or PPAR-delta agonists, cholesterol absorption inhibitors, polymeric bile acid adsorbers, bile acid reabsorption inhibitors, lipase inhibitors and lipoprotein (a) antagonists.

In a preferred embodiment of the invention, the dosage forms of the invention are administered in combination with a CETP inhibitor, for example and preferably torcetrapib (CP-5294/4), JJT-705 or CETP vaccine (Avant).

In a preferred embodiment of the invention, the dosage forms of the invention are administered in combination with a thyroid receptor agonist, for example and preferably D-thyroxine, 3,5,3′-triiodothyronine (T3), CGS 23425 or axitirome (CGS 26214).

In a preferred embodiment of the invention, the dosage forms of the invention are administered in combination with an HMG-CoA reductase inhibitor from the class of statins, for example and preferably lovastatin, simvastatin, pravastatin, fluvastatin, atorvastatin, rosuvastatin or pitavastatin.

In a preferred embodiment of the invention, the dosage forms of the invention are administered in combination with a squalene synthesis inhibitor, for example and preferably BMS-188494 or TAK-475.

In a preferred embodiment of the invention, the dosage forms of the invention are administered in combination with an ACAT inhibitor, for example and preferably avasimibe, melinamide, pactimibe, eflucimibe or SMP-797.

In a preferred embodiment of the invention, the dosage forms of the invention are administered in combination with an MTP inhibitor, for example and preferably implitapide, BMS-201038, R-103757 or JTT-130.

In a preferred embodiment of the invention, the dosage forms of the invention are administered in combination with a PPAR-gamma agonist, for example and preferably pioglitazone or rosiglitazone.

In a preferred embodiment of the invention, the dosage forms of the invention are administered in combination with a PPAR-delta agonist, for example and preferably GW 501516 or BAY 68-5042.

In a preferred embodiment of the invention, the dosage forms of the invention are administered in combination with a cholesterol absorption inhibitor, for example and preferably ezetimibe, tiqueside or pamaqueside.

In a preferred embodiment of the invention, the dosage forms of the invention are administered in combination with a lipase inhibitor, for example and preferably orlistat.

In a preferred embodiment of the invention, the dosage forms of the invention are administered in combination with a polymeric bile acid adsorber, for example and preferably cholestyramine, colestipol, colesolvam, CholestaGel or colestimide.

In a preferred embodiment of the invention, the dosage forms of the invention are administered in combination with a bile acid reabsorption inhibitor, for example and preferably ASBT (=IBAT) inhibitors, for example AZD-7806, S-8921, AK-105, BARI-1741, SC-435 or SC-635.

In a preferred embodiment of the invention, the dosage forms of the invention are administered in combination with a lipoprotein (a) antagonist, for example and preferably gemcabene calcium (CI-1027) or nicotinic acid.

In a preferred embodiment of the invention, the dosage forms of the invention are administered in combination with an acetylcholinesterase inhibitor, for example and preferably donepezil, galantamine or rivastigmine.

In a preferred embodiment of the invention, the dosage forms of the invention are administered in combination with a NMDA receptor antagonist, for example and preferably memantine.

In a preferred embodiment of the invention, the dosage forms of the invention are administered in combination with sGC stimulators, for example and preferably riociguat, nelociguat, vericiguat, praliciguat (IW-1973) or olinciguat (IW-1701).

In a preferred embodiment of the invention, the dosage forms of the invention are administered in combination with antidiabetics, for example and preferably metformin.

In a preferred embodiment of the invention, the dosage forms of the invention are administered in combination with SGLT-2 inhibitors, for example and preferably dapagliflozin, empagliflozin, canagliflozin, ipragliflozin and/or tofogliflozin.

In a preferred embodiment of the invention, the dosage forms of the invention are administered in combination with substances for the treatment of bone disorders, for example and preferably vitamin D or metabolites thereof, strontium ranelate, selective oestrogen receptor modulators (SERM) and/or RANKL modulators.

In a preferred embodiment of the invention, the dosage forms of the invention are administered in combination with bisphosphonates, for example and preferably etidronate, clodronate, tiludronate, teriparatide, pamidronate, neridronate, olpadronate, alendronate, ibandronate, risedronate, zoledronate.

The invention further provides for the use of an osmotic release system of the invention comprising the compound of the formula (I) for the treatment and/or prevention of diseases.

The invention further provides for the use of an osmotic release system of the invention comprising the compound of the formula (I) for the treatment and/or prevention of renal and cardiorenal disorders, in particular chronic kidney disease (CKD) and diabetic kidney disease (DKD), cardiac and cardiovascular disorders, in particular heart failure (HFpEF and HFrEF), myocardial infarction, angina pectoris, cardiomyopathies, hypertension and arteriosclerosis, pulmonary and cardiopulmonary disorders, in particular pulmonary hypertension (PH), disorders of the central nervous system, in particular dementia, bone disorders, in particular osteogenesis imperfecta, thromboembolic disorders, muscular dystrophies, ischaemias, vascular disorders, microcirculation impairment, fibrotic disorders, in particular systemic sclerosis, ophthalmological disorders, inflammatory disorders, and metabolic disorders, in particular metabolic syndrome, dyslipidaemia and diabetes.

The invention further provides for the use of an osmotic release system of the invention comprising the compound of the formula (I) for the treatment and/or prevention of renal and cardiorenal disorders, in particular chronic kidney disease (CKD) and diabetic kidney disease (DKD).

The invention further provides for the use of an osmotic release system of the invention comprising the compound of the formula (I) for the treatment and/or prevention of cardiac and cardiovascular disorders, in particular heart failure (HFpEF and HFrEF), myocardial infarction, angina pectoris, cardiomyopathies, hypertension and arteriosclerosis.

The invention further provides for the use of an osmotic release system of the invention comprising the compound of the formula (I) for the treatment and/or prevention of pulmonary and cardiopulmonary disorders, in particular pulmonary hypertension (PH).

The invention further provides for the use of an osmotic release system of the invention comprising the compound of the formula (I) for the treatment and/or prevention of disorders of the central nervous system, in particular dementia.

The invention further provides for the use of an osmotic release system of the invention comprising the compound of the formula (I) for the treatment and/or prevention of disorders of the central nervous system, in particular vascular and Alzheimer's dementia.

The invention further provides for the use of an osmotic release system of the invention comprising the compound of the formula (I) for the treatment and/or prevention of metabolic disorders, in particular metabolic syndrome, dyslipidaemia and diabetes.

The invention further provides for the use of an osmotic release system of the invention comprising the compound of the formula (I) as described above in combination with one or more other active substances selected from the group consisting of organic nitrates, NO donors, cGMP-PDE inhibitors, stimulators of guanylate cyclase, antithrombotics, antihypertensives, MR antagonists, IP receptor agonists, anti-inflammatory active substances, antidementia drugs, antidiabetics, active substances that modify fat metabolism and active substances for the treatment of bone and muscle disorders.

In the dosage forms of the invention, the compound of the formula (I) is preferably present in an amount of about 1 to 240 mg, more preferably in an amount of about 1 mg to 120 mg, most preferably in an amount of about 2.5 mg to 50 mg. The present invention provides the abovementioned pharmaceutical dosage forms of the invention comprising the compound of the formula (I) preferably in an amount of 1 mg, 2 mg, 2.5 mg, 3 mg, 4 mg, 5 mg, 6 mg, 7 mg, 8 mg, 9 mg, 10 mg, 12 mg, 15 mg, 20 mg, 25 mg, 30 mg, 35 mg, 40 mg, 50 mg, 60 mg, 70 mg, 75 mg, 80 mg, 90 mg, 100 mg, 110 mg, 120 mg, 125 mg, 150 mg, 175 mg, 200 mg, 225 mg and 240 mg. The amounts of the compound of the formula (I) refer to the nominal amounts in the pharmaceutical dosage form, in certain circumstances an excess of up to 20% of the amount of active ingredient may additionally be present.

In general, it has been found to be advantageous to administer about 0.01 to 10 mg/kg body weight per day to achieve effective results.

It may nevertheless be necessary in some cases to depart from the stated amounts, specifically as a function of body weight, route of administration, individual response to the active ingredient, nature of the formulation and time at which or interval over which administration takes place. Thus in some cases it may be sufficient to manage with less than the abovementioned minimum amount, whereas in other cases it is necessary to exceed the cited upper limit. If administering larger amounts, it may be advisable to divide them into several individual doses over the day.

### EXPERIMENTAL

Dissolution Profile

The dissolution of the active ingredient from the tablets is determined by the US Pharmacopoeia (USP 39) method (Chapter <711> Dissolution) using Apparatus 2 (paddle test). For the determination of the dissolution rate, a tablet is introduced into each receptacle of USP Apparatus 2 and the amount of active ingredient that has gone into solution after the undissolved constituents have been filtered off is determined by HPLC. The dissolution medium used is phosphate buffer pH 6.8 without addition of surfactant, and the paddle stirrer of the USP Apparatus 2 has a speed of rotation of 100 revolutions per minute. Unless otherwise stated, the dissolution rate of at least six test specimens is determined. In each case, the average amount of active ingredient released is reported.

Thermoanalytical Investigation of Binary Physical Mixtures

In order to illustrate compatibilities in thermoanalytical investigations, a mortar was charged with equal parts of the compound of the formula (I) and of hydrophilic swellable polymers and the contents were ground with a pestle into a homogeneous powder mixture (trituration in a ratio of 1:1, binary mixture). The hydrophilic swellable polymers investigated were polyethylene oxide (meeting the requirements of the Ph. Eur. (9th edition) monograph “Macrogols, High Molecular Mass”; viscosity from 40 to 100 mPa·s; measured in 5% aqueous solution 25° C.; POLYOX™ water-soluble resin NF WSR N-80; Dow), xanthan (“Xanthan FN food grade, normal” produced by Jungbunzlauer Ladenburg GmbH) meeting the requirements of the Ph. Eur. (9th edition) monograph “Xanthan gum”, vinylpyrrolidone-vinyl acetate copolymer (Kollidon VA 64) meeting the requirements of the Ph. Eur. (9th edition) monograph “Copovidone”, polyvinylpyrrolidone (PVP 25) meeting the requirements of the Ph. Eur. (9th edition) monograph “Povidone”, methacrylic acid-methyl methacrylate copolymer (Eudragit® L100) meeting the requirements of the Ph. Eur. (9th edition) monograph “Methacrylic acid-Methyl Methacrylate Copolymer (1:1)”, methacrylic acid-methyl methacrylate copolymer (Eudragit® RL PO) meeting the requirements of the Ph. Eur. (9th edition) monograph “Ammonio Methacrylate Copolymer (Type A)”, hydroxypropylcellulose (HPC LM Nisso) meeting the requirements of the Ph. Eur. (9th edition) monograph “Hydroxypropylcellulose” and polyacrylic acid (meeting the requirements of the Ph. Eur. (9th edition) monograph “Carbomers”; designation: Polyacrylic acid, MW 1 080 000, aver. MN 135,000; Acros Organics).

The physical mixtures and the respective individual components were characterized thermoanalytically.

The thermograms were recorded on a DSC (differential scanning calorimeter). For this, about 5 mg of sample was in each case heated in an aluminium crucible under nitrogen (50 ml/min) at a heating rate of 10 K/min until the end of the melting point of the respective compound.

Unless specified more precisely, the substances used refer to the pharmaceutical excipients known to those skilled in the art under the name cited and, if listed in the respective pharmacopoeia, meet the respective requirements of the pharmacopoeial monographs of the European (Ph. Eur 9), US (USP 41 and NF 36) and/or Japanese (JP, 17th edition) pharmacopoeias.

FIG. 1 shows thermograms of the compound of the formula (I), of polyethylene oxide and of binary mixtures of the compound of the formula (I) with polyethylene oxide.

FIG. 2 shows thermograms of the compound of the formula (I), of xanthan and of binary mixtures of the compound of the formula (I) with xanthan.

FIG. 3 shows thermograms of the compound of the formula (I), of vinylpyrrolidone-vinyl acetate copolymer and of binary mixtures of the compound of the formula (I) with vinylpyrrolidone-vinyl acetate copolymer.

FIG. 4 shows thermograms of the compound of the formula (I), of PVP 25 and of binary mixtures of the compound of the formula (I) with PVP 25.

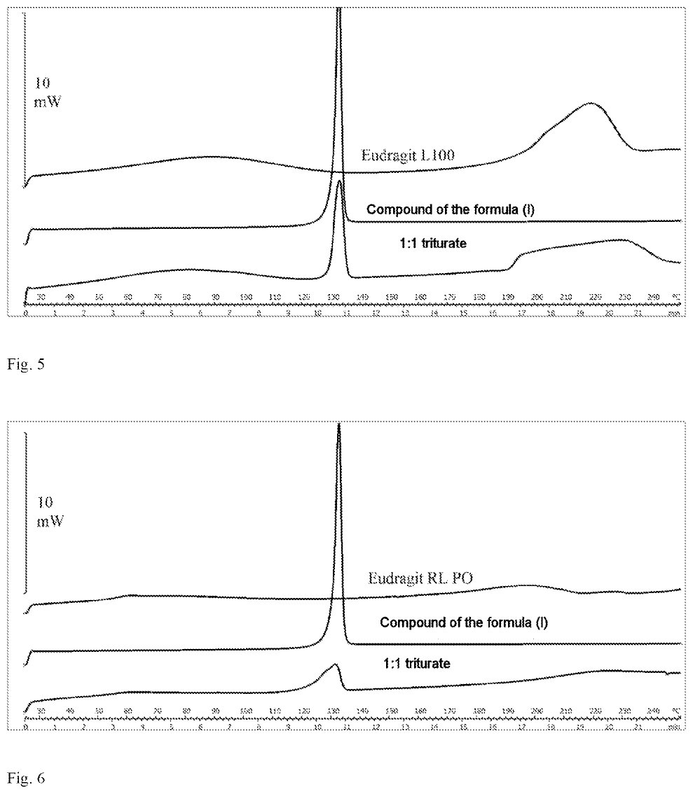

FIG. 5 shows thermograms of the compound of the formula (I), of Eudragit L100 and of binary mixtures of the compound of the formula (I) with Eudragit L100.

FIG. 6 shows thermograms of the compound of the formula (I), of Eudragit RL PO and of binary mixtures of the compound of the formula (I) with Eudragit RL PO.

FIG. 7 shows thermograms of the compound of the formula (I), of HPC LM and of binary mixtures of the compound of the formula (I) with HPC LM.

FIG. 8 shows thermograms of the compound of the formula (I), of polyacrylic acid and of binary mixtures of the compound of the formula (I) with polyacrylic acid.

1. Osmotic release system consisting of a core and a shell, wherein the shell consists of a water-permeable material impermeable to the components of the core and has at least one orifice and wherein the core comprises (3S)-3-(4-chloro-3-{[(2S,3R)-2-(4-chlorophenyl)-4,4,4-trifluoro-3-methylbutanoyl]amino}phenyl)-3-cyclopropylpropanoic acid of the formula (I) 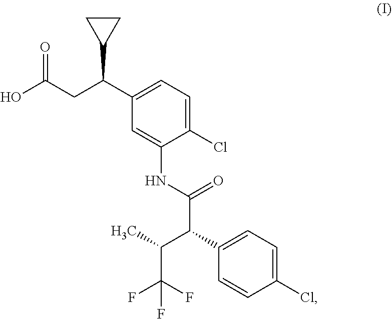 [SMILES: C[C@H]([C@H](C(=O)Nc1cc([C@@H](CC(=O)O)C2CC2)ccc1Cl)c1ccc(Cl)cc1)C(F)(F)F]
and at least one hydrophilic swellable polymer, wherein the hydrophilic swellable polymer is not polyethylene oxide.

2. Osmotic release system according to claim 1, wherein the core of the osmotic release system comprises
0.5% by weight to 50% by weight of the compound of the formula (I),
40% by weight to 99.5% by weight of at least one hydrophilic swellable polymer
and optionally at least one osmotically active additive and optionally at least one pharmaceutically customary excipient.

3. Osmotic release system according to claim 1, wherein the core comprises
0.5% by weight to 50% by weight of the compound of the formula (I),
10% by weight to 50% by weight of xanthan,
5% by weight to 40% by weight of a vinylpyrrolidone-vinyl acetate copolymer,
optionally at least one further hydrophilic swellable polymer, optionally at least one further pharmaceutically customary excipient and optionally at least one osmotically active additive.

4. Osmotic release system according to claim 1, wherein the core comprises a two-chamber system consisting of an active ingredient layer and an osmosis layer.

5. Osmotic release system according to claim 4, wherein the active ingredient layer comprises
1% by weight to 50% by weight of the compound of the formula (I),
20% by weight to 99% by weight of at least one hydrophilic swellable polymer,
and optionally at least one osmotically active additive and optionally at least one pharmaceutically customary excipient,
and the osmosis layer comprises
40% by weight to 90% by weight of at least one hydrophilic swellable polymer,
10% by weight to 60% by weight of an osmotically active additive,
and optionally at least one pharmaceutically customary excipient.

6. Osmotic release system according to claim 1, wherein at least one hydrophilic swellable polymer is selected from a list consisting of xanthan, cellulose derivatives, for example hydroxypropylcellulose, hydroxypropyl methylcellulose or sodium carboxymethylcellulose, starch derivatives, for example sodium carboxymethyl starch, vinylpyrrolidone-vinyl acetate copolymer, polyvinylpyrrolidone, methacrylic acid copolymers, for example methacrylic acid-methyl methacrylate copolymer and polyacrylic acids.

7. Osmotic release system according to claim 1, wherein the shell consists of cellulose acetate or a mixture of cellulose acetate and polyethylene glycol.

8. Process for producing an osmotic release system according to claim 1, characterized in that the components of the core are mixed with one another, granulated and tableted, the resulting core is coated with a shell and the shell is then provided with one or more orifices suitable for the escape of the compound of the formula (I).

9. Process for producing an osmotic release system according to claim 4, characterized in that
the components of the active ingredient layer are mixed and granulated and
the components of the osmosis layer are mixed and granulated,
the two granulates are then pressed on a bilayer tablet press to obtain a bilayer tablet,
the resulting core is then coated with the shell and
the shell is, on the active ingredient side, provided with one or more orifices.

10. Osmotic release system according to claim 1 for the treatment and/or prevention of diseases.

11. Osmotic release system according to claim 1 for the treatment and/or prevention of renal and cardiorenal disorders, in particular chronic kidney disease (CKD) and diabetic kidney disease (DKD), cardiac and cardiovascular disorders, in particular heart failure (HFpEF and HFrEF), myocardial infarction, angina pectoris, cardiomyopathies, hypertension and arteriosclerosis, pulmonary and cardiopulmonary disorders, in particular pulmonary hypertension (PH), ophthalmological disorders, in particular non-proliferative diabetic retinopathy (NPDR) and diabetic macular oedema (DMO), disorders of the central nervous system, in particular dementia, bone disorders, in particular osteogenesis imperfecta, thromboembolic disorders, muscular dystrophies, ischaemias, vascular disorders, microcirculation impairment, fibrotic disorders, in particular systemic sclerosis, inflammatory disorders, and metabolic disorders, in particular metabolic syndrome, dyslipidaemia and diabetes.

12. Osmotic release system according to claim 1 in combination with one or more other active ingredients selected from the group consisting of organic nitrates, NO donors, cGMP-PDE inhibitors, stimulators of guanylate cyclase, antithrombotics, antihypertensives, MR antagonists, IP receptor agonists, anti-inflammatory active substances, antidementia drugs, antidiabetics, active substances that modify fat metabolism and active substances for the treatment of bone and muscle disorders.

13. Method for the treatment and/or prevention of renal and cardiorenal disorders, in particular chronic kidney disease (CKD) and diabetic kidney disease (DKD), cardiac and cardiovascular disorders, in particular heart failure (HFpEF and HFrEF), myocardial infarction, angina pectoris, cardiomyopathies, hypertension and arteriosclerosis, pulmonary and cardiopulmonary disorders, in particular pulmonary hypertension (PH), ophthalmological disorders, in particular non-proliferative diabetic retinopathy (NPDR) and diabetic macular oedema (DMO), disorders of the central nervous system, in particular dementia, bone disorders, in particular osteogenesis imperfecta, thromboembolic disorders, muscular dystrophies, ischaemias, vascular disorders, microcirculation impairment, fibrotic disorders, in particular systemic sclerosis, inflammatory disorders, and metabolic disorders, in particular metabolic syndrome, dyslipidaemia and diabetes in humans and animals comprising administering a therapeutically effective amount of the osmotic release system according to claim 1 to a human or animal in need thereof.
